(19) United States
(12) Patent Application Publication
(10) Pub. No.: US 2018/0244726 A1
(43) Pub. Date: Aug. 30, 2018

(54) Fusion Protein

(71) Applicant: Viravaxx AG, Vienna (AT)

(72) Inventors: Rudolf Valenta, Theresienfeld (AT); Carolin Cornelius, Vienna (AT)

(73) Assignee: Viravaxx AG, Vienna (AT)

(21) Appl. No.: 15/756,514

(22) PCT Filed: Sep. 5, 2016
(86) PCT No.: PCT/EP2016/070824

§ 371 Date: Feb. 28, 2018

(30) Foreign Application Priority Data
Sep. 5, 2015 (EP) 15183983.4

(51) Int. Cl. C07K 14/005

(52) U.S. Cl. CPC C07K 14/005; C12N 2730/10134; C07K 2319/00

(57) ABSTRACT

The present invention relates to a fusion protein for use in the treatment and/or prevention of a hepatitis B virus infection comprising at least one hepatitis B PreS polypeptide or fragment thereof fused to at least one peptide consisting of an amino acid sequence having at least 80% identity to a sequence selected from the group consisting of SEQ ID No. 1, SEQ ID No. 2, SEQ ID No. 3 and SEQ ID No. 4.

The present invention relates to a fusion protein for use in the treatment and/or prevention of a hepatitis B virus (HBV) infection.

Hepatitis B is a liver disease caused by hepatitis B viruses. The disease affects millions of people per year throughout the world. The HBV is present in the blood and body fluids of infected people and can therefore be spread by contacting these fluids with fluids of healthy people.

HBV primarily interferes with the functions of the liver by replicating in liver cells. During HBV infection, the host immune response causes both hepatocellular damage and viral clearance.

Acute HBV infections are usually not treated because most people are able to clear the infection spontaneously. However, chronic HBV infections have to be treated in order to reduce the risk of cirrhosis and liver cancer. Antiviral drugs currently used in the treatment of HBV infections include lamivudine, adefovir, tenofovir, telbivudine and entecavir. Furthermore, interferon alpha-2a acting as immune system modulator can also be used in the treatment. However, none of these drugs can clear HBV infections. These drugs can only stop the HBV from replicating, thus minimizing liver damage.

It is an object of the present invention to provide new means in the treatment and/or prevention of HBV infections which overcome the drawbacks of the present HBV treatments. A particularly important objective is viral clearance of HBV in chronically infected patients by restoring an efficient humoral and cellular immune response.

These objectives are achieved by a fusion protein for use in the treatment and/or prevention of a hepatitis B virus infection comprising at least one hepatitis B PreS polypeptide or fragment thereof fused to at least one peptide consisting of an amino acid sequence having at least 80% identity to a sequence selected from the group consisting of SEQ ID No. 1, SEQ ID No. 2, SEQ ID No. 3 and SEQ ID No. 4.

It surprisingly turned out that a fusion protein comprising a hepatitis B PreS polypeptide or fragment thereof and at least one peptide consisting of an amino acid sequence having at least 80% identity to a sequence selected from the group consisting of SEQ ID No. 1, SEQ ID No. 2, SEQ ID No. 3 and SEQ ID No. 4 induces the formation of PreS specific antibodies in an individual to a much higher extent compared to PreS alone or other fusion proteins comprising peptides different from SEQ ID No. 1, SEQ ID No. 2, SEQ ID No. 3 and SEQ ID No. 4. Furthermore the antibodies produced in response to the administration of the fusion protein of the present invention show superior hepatitis B neutralizing effects and are able to inhibit hepatitis B virus infections. This is the first time that the administration of a fusion protein comprising PreS can be successfully be used in the treatment and/or prevention of a hepatitis B virus infection in a human subject.

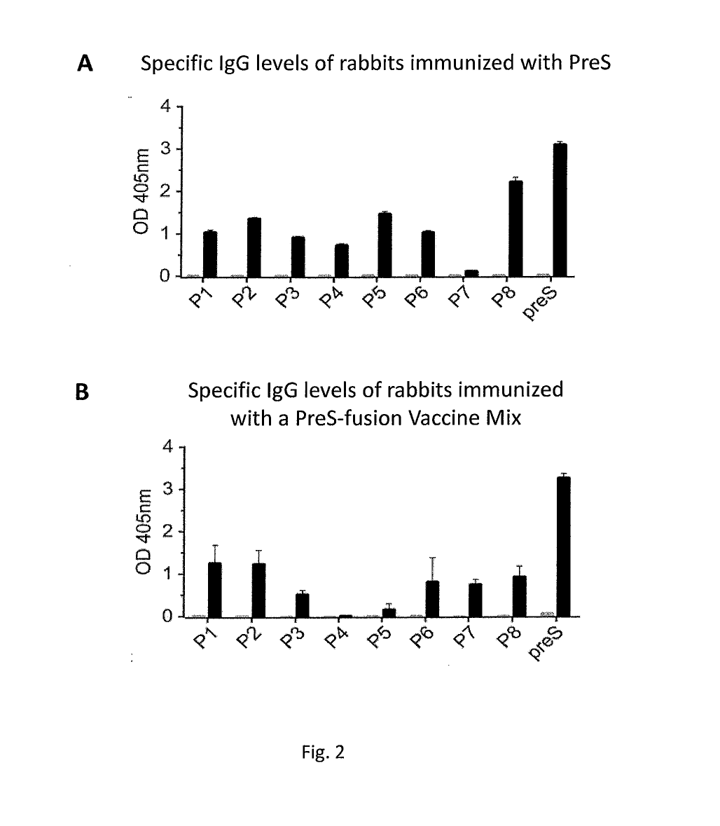

As shown in FIG. 2B the administration of the fusion protein of the present invention results in the formation of antibodies which are specifically directed to the first 30 (peptide P1) and 50 (peptide P2) amino acid residues of HBV PreS, to a lower extent to the C-terminal region (peptides P6 to P8) and to a negligible extent to the central part of HBV PreS (peptides P4 and P5). Since the N-terminal part of HBV PreS is known to play an important role in liver cell attachment of HBV and HBV infections antibodies directed to this part of the PreS polypeptide are particularly useful in the treatment and/or prevention of HBV infections. In contrast thereto, the sole administration of HBV PreS does not show these effects. The antibodies produced thereby are able to bind to almost any part of HBV PreS (see FIG. 2A). This shows that the immune response induced by the fusion proteins of the present invention is more focused on those parts of the HBV PreS polypeptide which are involved in the HBV infection.

The fusion protein of the present invention may comprise one or more hepatitis B PreS polypeptides or one or more fragments thereof. The presence of more than one hepatitis B PreS polypeptides or fragments thereof in the fusion protein has the advantage that more antigens are presented to the immune system allowing the formation of even more antibodies directed to PreS. In a particularly preferred embodiment of the present invention the fusion protein comprises one, two, three, four, five, six, seven, eight, nine or ten hepatitis B PreS polypeptides or fragments thereof. The HBV PreS polypeptides as well as their fragments as defined herein being part of the fusion protein of the present invention may be derived from the same HBV genotype or from different genotypes. For instance, the fusion protein of the present invention may comprise the PreS polypeptide or a fragment thereof of HBV genotype A only or may be combined with a further PreS polypeptide or fragment thereof derived from HBV genotype B, C, D, E, F, G or H.

In a particularly preferred embodiment of the present invention the fusion protein comprises at least one peptide consisting of an amino acid sequence having at least 80% identity to SEQ ID No. 1, at least one peptide consisting of an amino acid sequence having at least 80% identity to SEQ ID No. 2, at least one peptide consisting of an amino acid sequence having at least 80% identity to SEQ ID No. 3 and at least one peptide consisting of an amino acid sequence having at least 80% identity to SEQ ID No. 4. Alternatively the fusion protein of the present invention may comprise one, two, three, four, five six, seven, eight, nine or ten of these peptides in any possible combination or even only one specific peptide in the same amount.

The terms “fused to” or “fusion protein”, as used herein, refer to a protein comprising a hepatitis B PreS polypeptide or fragment thereof that are expressed and prepared as one single recombinant polypeptide chain.

Methods for the production of fusion proteins are well known in the art and can be found in standard molecular biology references such as Sambrook et al. (Molecular Cloning, 2nd ed., Cold Spring Harbor Laboratory Press, 1989) and Ausubel et al. (Short Protocols in Molecular Biology, 3rd ed; Wiley and Sons, 1995). In general, a fusion protein is produced by first constructing a fusion gene which is inserted into a suitable expression vector, which is, in turn, used to transfect a suitable host cell. In general, recombinant fusion constructs are produced by a series of restriction enzyme digestions and ligation reactions which result in the desired sequences being incorporated into a plasmid. If suitable restriction sites are not available, synthetic oligonucleotide adapters or linkers can be used as is known by those skilled in the art and described in the references cited above. The polynucleotide sequences encoding allergens and native proteins can be assembled prior to insertion into a suitable vector or the sequence encoding the allergen can be inserted adjacent to a sequence encoding a native sequence already present in a vector. Insertion of the sequence within the vector should be in frame so that the sequence can be transcribed into a protein. It will be apparent to those of ordinary skill in the art that the precise restriction enzymes, linkers and/or adaptors required as well as the precise reaction conditions will vary with the sequences and cloning vectors used. The assembly of DNA constructs, however, is routine in the art and can be readily accomplished by a person skilled in the art.

A fragment of a hepatitis B PreS polypeptide consists preferably of at least 30, preferably at least 40, more preferably at least 50, consecutive amino acid residues and may comprise PreS1 and/or PreS2 of the hepatitis B PreS polypeptide. In a particularly preferred embodiment of the present invention a fragment of a hepatitis B PreS polypeptide may comprise amino acid residues 1 to 70, preferably amino acid residues 1 to 65, more preferably amino acid residues 1 to 60, more preferably amino acid residues 1 to 55, more preferably amino acid residues 1 to 50, more preferably 1 to 45, more preferably amino acid residues 1 to 40, more preferably amino acid residues 1 to 35, more preferably amino acid residues 5 to 70, more preferably amino acid residues 5 to 65, more preferably amino acid residues 5 to 60, more preferably amino acid residues 5 to 55, more preferably amino acid residues 5 to 50, more preferably 5 to 45, more preferably amino acid residues 5 to 40, more preferably amino acid residues 5 to 35, more preferably amino acid residues 10 to 70, more preferably amino acid residues 10 to 65, more preferably amino acid residues 10 to 60, more preferably amino acid residues 10 to 55, more preferably amino acid residues 10 to 50, more preferably 10 to 45, more preferably amino acid residues 10 to 40, more preferably amino acid residues 10 to 35, more preferably amino acid residues 15 to 70, more preferably amino acid residues 15 to 65, more preferably amino acid residues 15 to 60, more preferably amino acid residues 15 to 55, more preferably amino acid residues 15 to 50, more preferably 15 to 45, more preferably amino acid residues 15 to 40, more preferably amino acid residues 15 to 35, of the hepatitis B PreS polypeptide, preferably of the HBV PreS polypeptides consisting of SEQ ID Nos. 5, 7, 8, 9, 10, 11, 12, 13 or 14, whereby SEQ ID Nos. 8 to 14 belong to HBV genotypes B to H, respectively.

The at least one peptide to be fused to at least one hepatitis B PreS polypeptide or fragment thereof has an identity of at least 80%, preferably of at least 85%, more preferably of at least 90%, more preferably of at least 92%, more preferably of at least 94%, more preferably of at least 96%, more preferably of at least 98%, more preferably of at least 99%, in particular of 100%, to SEQ ID No. 1, SEQ ID No. 2, SEQ ID No. 3 and SEQ ID No. 4. The degree of identity of a first amino acid sequence to a second amino acid can be determined by a direct comparison between both amino acid sequences using certain algorithms. Sequence identity is preferably determined by BLAST alignment (http://blast.ncbi.nlm.nih.gov/; Altschul S F et al J. Mol. Biol. 215 (1990): 403-410) using the BLOSUM62 matrix, a gap existence penalty of 11, and a gap extension penalty of 1.

According to a preferred embodiment of the present invention the amino acid sequence of the PreS polypeptide is at least 80% identical to SEQ ID No. 5, SEQ ID No. 7, SEQ ID No. 8, SEQ ID No. 9, SEQ ID No. 10, SEQ ID No. 11, SEQ ID No. 12 or SEQ ID No. 13, most preferably to SEQ ID No. 5.

The hepatitis B PreS polypeptide to be fused to at least one of the peptides described above has an identity of at least 80%, preferably of at least 85%, more preferably of at least 90%, more preferably of at least 92%, more preferably of at least 94%, more preferably of at least 96%, more preferably of at least 98%, more preferably of at least 99%, in particular of 100%, to SEQ ID No. 5, SEQ ID No. 7, SEQ ID No. 8, SEQ ID No. 9, SEQ ID No. 10, SEQ ID No. 11, SEQ ID No. 12 or SEQ ID No. 13.

According to a further preferred embodiment of the present invention the at least one peptide is fused to the N- and/or C-terminus of the PreS polypeptide.

“Fused to the N- and/or C-terminus”, as used herein, means that at least one peptide is fused to the N- and/or C-terminus of the PreS polypeptide or fragment thereof. The fusion protein of the present invention may comprise one or more peptides fused to the N-terminus of the PreS polypeptide or fragment thereof or to its C-terminus.

According to a preferred embodiment of the present invention the fusion protein comprises an amino acid sequence which is at least 80% identical to SEQ ID No. 6.

The fusion protein of the present invention has an identity of at least 80%, preferably of at least 85%, more preferably of at least 90%, more preferably of at least 92%, more preferably of at least 94%, more preferably of at least 96%, more preferably of at least 98%, more preferably of at least 99%, in particular of 100%, to SEQ ID No. 6.

According to a further preferred embodiment of the present invention the hepatitis B virus infection is caused by a hepatitis B virus genotype A, B, C, D, E, F, G, H or a subtype therof. It is preferred to use a HBV PreS polypeptide or fragment thereof of the same genotype to treat and/or prevent a HBV caused by this HBV genotype (e.g. PreS of HBV genotype A is used to treat/prevent an infection of HBV genotype A or one of its subtypes). Due to the conserved amino acid sequences in those parts of the PreS polypeptide which is known to be involved in the HBV infection, it is of course also possible to use a HBV PreS polypeptide or fragment thereof of one genotype to treat/prevent an infection of another HBV genotype (e.g. PreS of HBV genotype A is used to treat/prevent an infection of HBV genotype B, C, D, E, F, G and/or H or a subtype thereof).

The fusion protein of the present invention may be used in the treatment and/or prevention of HBV infections of various genotypes and subtypes thereof. Subtypes of hepatitis B viruses include A1, A2, A3, A4, A5, B1, B2, B3, B4, B5, C1, C2, C3, C4, C5, D1, D2, D3, D4, D5, F1, F2, F3 and F4 as discussed in Schaefer et al. (World J Gastroenterol 13(2007):14-21).

According to a particularly preferred embodiment of the present invention the fusion protein is administered to an individual at least once in an amount of 0.01 μg/kg body weight to 5 mg/kg body weight, preferably 0.1 μg/kg body weight to 2 mg/kg body weight. According to further preferred embodiment of the present invention the fusion protein is administered to a patient in an amount of 5 to 50 μg, preferably 10 to 40 μg, more preferably 15 to 30 μg, either independent from the body weight (i.e. a dose may comprise 15, 20, 25 or 30 μg) or per kg body weight.

The amount of fusion protein that may be combined with excipients to produce a single dosage form will vary depending upon the host treated and the particular mode of administration. The dose of the fusion protein may vary according to factors such as the disease state, age, sex and weight of the individual, and the ability to elicit the desired antibody response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances. For instance, the polypeptides and vaccine of the present invention may be administered to an individual at intervals of several days, one or two weeks or even months depending always on the level of hepatitis B PreS specific IgG induction.

In a preferred embodiment of the present invention the fusion protein of the present invention is applied between 2 and 10, preferably between 2 and 7, even more preferably up to 5 and most preferably up to 3 times. In a particularly preferred embodiment the time interval between the subsequent vaccinations is chosen to be between 2 weeks and 5 years, preferably between 1 month and up to 3 years, more preferably between 2 months and 1.5 years. The repeated administration of the fusion protein of the present invention may maximize the final effect of the treatment.

According to a further preferred embodiment of the present invention the fusion protein is administered together with at least one adjuvant and/or pharmaceutical acceptable excipient.

The fusion protein of the present invention can be administrated subcutaneously, intramuscularly, intravenously, mucosally etc. Depending on the dosage form and administration route the fusion protein of the present invention may be combined with excipients, diluents, adjuvants and/or carriers. A preferred adjuvant is alum. Suitable protocols for the production of vaccine formulations are known to the person skilled in the art and can be found e.g. in “Vaccine Protocols” (A. Robinson, M. P. Cranage, M. Hudson; Humana Press Inc., U. S.; 2nd edition 2003).

The fusion protein of the present invention may be formulated also with other adjuvants regularly used in vaccines. For instance, suitable adjuvants may be MF59, aluminum phosphate, calcium phosphate, cytokines (e.g. IL-2, IL-12, GM-CSF), saponins (e.g. QS21), MDP derivatives, CpG oligonucleotides, LPS, MPL, polyphosphazenes, emulsions (e.g. Freund's, SAF), liposomes, virosomes, iscoms, cochleates, PLG microparticles, poloxamer particles, virus-like particles, heat-labile enterotoxin (LT), cholera toxin (CT), mutant toxins (e.g. LTK63 and LTR72), microparticles and/or polymerized liposomes. Suitable adjuvants are commercially available as, for example, AS01B (MPL and QS21 in a liposome formulation), AS02A, AS15, AS-2, AS-03 and derivatives thereof (GlaxoSmithKline, USA); CWS (cell-wall skeleton), TDM (trehalose-6,6′-dimycolate), LeIF (Leishmania elongation initiation factor), aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7 or -12 may also be used as adjuvants. Preferred adjuvants for use in eliciting a predominantly Th1-type response include, for example, a combination of monophosphoryl lipid A, preferably 3-O-deacylated monophosphoryl lipid A (3D-MPL), optionally with an aluminum salt. Aqueous formulations comprising monophosphoryl lipid A and a surfactant have been described in WO 98/43670.

Another preferred adjuvant is a saponin or saponin mimetics or derivatives, preferably QS21 (Aquila Biopharmaceuticals Inc.), which may be used alone or in combination with other adjuvants. For example, an enhanced system involves the combination of a monophosphoryl lipid A and saponin derivative, such as the combination of QS21 and 3D-MPL. Other preferred formulations comprise an oil-in-water emulsion and tocopherol. A particularly potent adjuvant formulation is QS21, 3D-MPL and tocopherol in an oil-in-water emulsion. Additional saponin adjuvants for use in the present invention include QS7 (described in WO 96/33739 and WO 96/11711) and QS17 (described in U.S. Pat. No. 5,057,540 and EP 0 362 279 B1).

The SEQ ID Nos. disclosed herein have the following amino acid sequence (Genbank Acc. No.:

| SEQ | | |
|---|---|---|
| ID | | Genbank |
| No. | Amino acid sequence | Acc. No. |
| 1 | EAAFNDAIKASTGGAYESYKFIPALEAAVK | |
| 2 | AEEVKVIPAGELQVIEKVDAAFKVAATAANAAPA | |
| | NDK | |
| 3 | ADLGYGPATPAAPAAGYTPATPAAPAEAAPAGK | |
| 4 | ATTEEQKLIEKINAGFKAALAAAAGVQPADKYR | |
| 5 | GGWSSKPRKGMGTNLSVPNPLGFFPDHQLDPAFG | AAT28735.1 |
| | ANSNNPDWDFNPIKDHWPAANQVGVGAFGPGLTP | |
| | PHGGILGWSPQAQGILTTVSTIPPPASTNRQSGR | |
| | QPTPISPPLRDSHPQAMQWNSTAFHQALQDPRVR | |
| | GLYFPAGGSSSGTVNPAPNIASHISSISARTGDP | |
| | VTN | |
| 6 | MEAAFNDAIKASTGGAYESYKFIPALEAAVKAEE | |
| | VKVIPAGELQVIEKVDAAFKVAATAANAAPANDK | |
| | GGWSSKPRKGMGTNLSVPNPLGFFPDHQLDPAFG | |
| | ANSNNPDWDFNPIKDHWPAANQVGVGAFGPGLTP | |
| | PHGGILGWSPQAQGILTTVSTIPPPASTNRQSGR | |
| | QPTPISPPLRDSHPQAMQWNSTAFHQALQDPRVR | |
| | GLYFPAGGSSSGTVNPAPNIASHISSISARTGDP | |
| | VTNADLGYGPATPAAPAAGYTPATPAAPAEAAPA | |
| | GKATTEEQKLIEKINAGFKAALAAAAGVQPADKY | |
| | R | |
| 7 | GGWSSKPRKGMGTNLSVPNPLGFFPDHQLDPAFK | BAE80758.1 |
| | ANSDNPDWDLNPHKDNWPDSNKVGVGAFGLGFTP | |
| | PHGGLLGWSPQAQGILTTVPAAPPPASTNRQVAR | |
| | PPTPLSPPLRDTHPQAMQWNSTTFHQTLQDPRVR | |
| | ALYFPAGGSSSGTVNPVQNTASSISSILSKTGDP | |
| | VPN | |
| 8 | GGYSSKPRKGMGTNLSVPNPLGFLPDHQLDPAFG | ADA56873.1 |
| | ANSNNPDWDFNPNKDPWPEAWQVGVGAFGPGFTP | |
| | PHGGLLGWSPQAQGILTPVPAAPPPASTNRQSGR | |
| | QPTPISPPLRDSHPQAMQWNSTTFHQALLDPRVR | |
| | GLYFPAGGSSSGTTNPVPTTASPISSIFSRTGDP | |
| | APN | |
| 9 | GQNLSTSNPLGFFPDHQLDPAFRANTANPDWDFN | AF121240.1 |
| | PNKDTWPDANKVGAGAFGLGFTPPHGGLLGWSPQ | |
| | AQGILQTLPTNPPPASTNRQSGRQPTPLSPPLRN | |
| | THPQAMQWNSTTFHQTLQDPRVRGLYFPAGGSSS | |
| | GTVNPVPTTVSHISSIFSRIGDPALN | |
| 10 | GLSWTVPLEWGKNHSTTNPLGFFPDHQLDPAFRA | AAW79851.1 |
| | NTRNPDWDHNPNKDHWTAANKVGVGAFGPGFTPP | |
| | HGGLLGWSPQAQGMLKTLPADPPPASTNRQSGRQ | |
| | PTPITPPLRDTHPQAMQWNSTTFHQALQDPRVRG | |
| | LYFPAGGSSSGTVNPVPTTASLISSIFSRIGDPA | |
| | PN | |
| 11 | GAPLSTTRRGMGQNLSVPNPLGFLPDHQLDPLFR | AAM09056.1 |
| | ANSSSPDWDFNKSKDNWPMANKVGVGGYGPGFTP | |
| | PHGGLLGWSPQAQGVLTTLPADPPPASTNRRSGR | |
| | KPTPVSPPLRDTHPQAMQWNSTQFHQALLDPRVR | |
| | ALYFPAGGSSSETQNPAPTIASLTSSIFLKTGGP | |
| | ANN | |
| 12 | GLSWTVPLEWGKNLSTSNPLGFLPDHQLDPAFRA | AB064313.1 |
| | NTNNPDWDFNPKKDPWPEANKVGVGAYGPGFTPP | |
| | HGGLLGWSPQSQGTLTTLPADPPPASTNRQSGRQ | |
| | PTPISPPLRDSHPQAMQWNSTAFHQALQNPKVRG | |
| | LYFPAGGSSSGIVNPVPTIASHISSIFSRIGDPA | |
| | PN | |
| 13 | GAPLSTARRGMGQNLSVPNPLGFFPDHQLDPLFR | FJ356716.1 |
| | ANSSSPDWDFNTNKDNWPMANKVGVGGFGPGFTP | |
| | PHGGLLGWSPQAQGILTTSPPDPPPASTNRRSGR | |
| | KPTPVSPPLRDTHPQAMQWNSTQFHQALLDPRVR | |
| | GLYFPAGGSSSETQNPAPTIASLTSSIFSKTGDP | |
| | ANN | |

The present invention is further illustrated by the following figures and examples, however, without being restricted thereto.

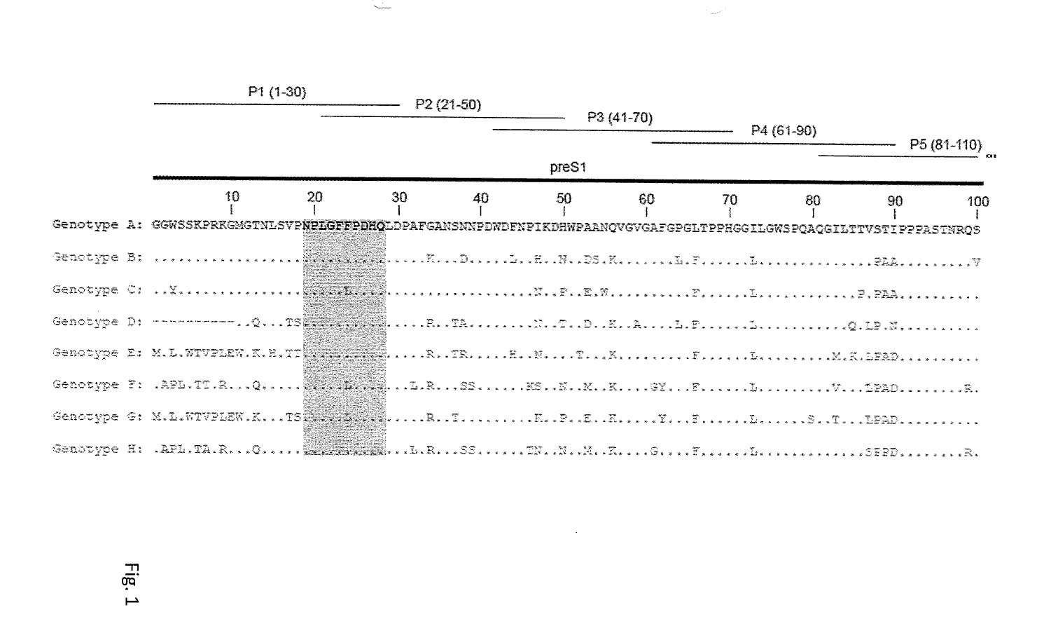

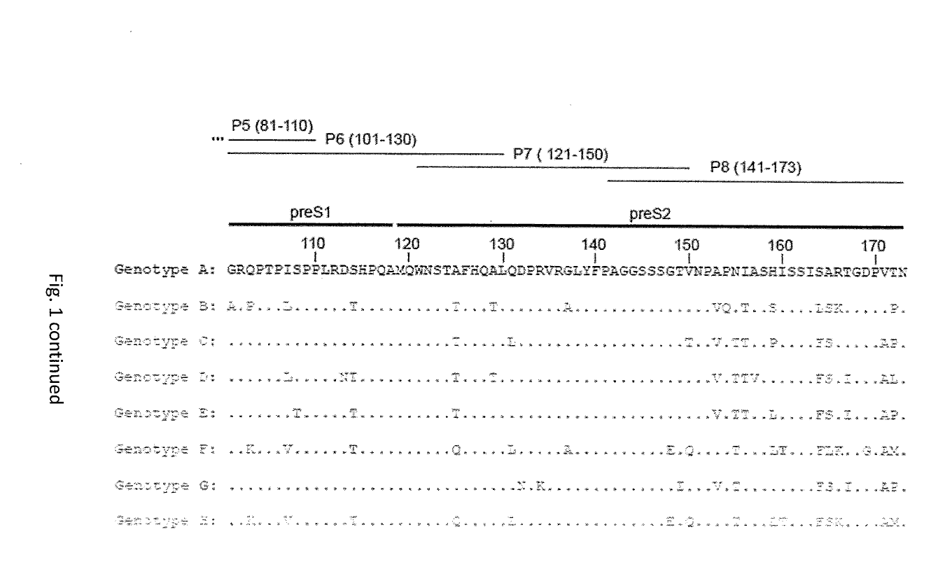

FIG. 1 shows the allocation of PreS peptides to aligned PreS sequences from different genotypes. Identical amino acids are indicated by points, the PreS1 domain includes amino acid residues 1 to 118 and the PreS2 domain amino acid residues 119 to 173 (see also SEQ ID No. 5) and amino acid residues 19 to 28 (grey box) play a crucial role in liver cell attachment of HBV and infection.

FIG. 2 shows IgG responses of rabbits immunized with PreS (n=1; FIG. 2A), or 20 μg of a PreS-fusion vaccine Mix (n=2; FIG. 2B), before (left bars in grey) and after (right bars in black) immunization. Optical density values (y-axes: OD values at 405 nm) correspond to IgG levels towards PreS and PreS-derived synthetic overlapping peptides P1-P8 (x-axes). Results represent mean values with SD from triplicate determinations.

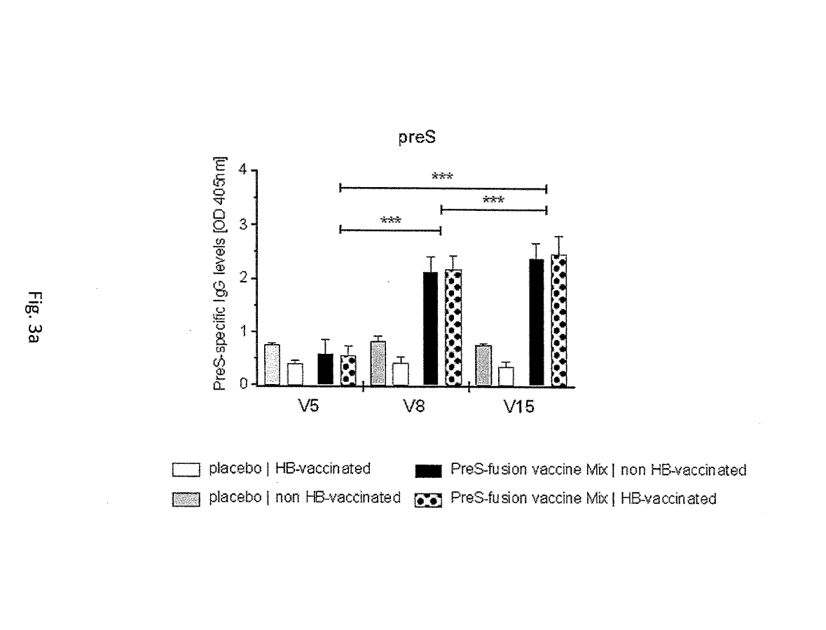

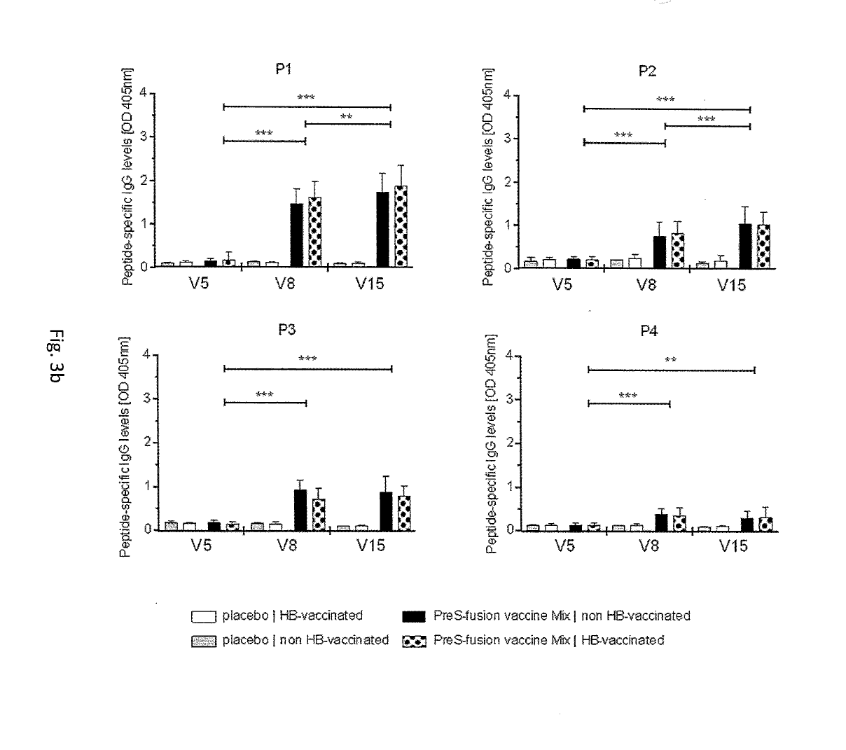

FIGS. 3A to 3C show IgG responses towards PreS (FIG. 3A) and synthetic PreS-derived overlapping peptides P1-P8 (FIGS. 3B, and 3C) of subjects vaccinated with PreS-fusion vaccine Mix or placebo. Shown are optical density values (y-axes: OD values, means of triplicate determinations) corresponding to IgG levels towards PreS and peptides P1-P8 measured in subjects with or without prior hepatitis B vaccination who had been immunized with PreS-fusion vaccine Mix (n=22) or placebo (n=8) before (V5) and at different time points after immunization (V8 and V15) (x-axes). Results are represented as mean values with SD and significant differences (in all PreS-fusion vaccine Mix-vaccinated individuals at V5, V8, and V15) are indicated: *p<0.05, **p<0.01, ***p<0.001.

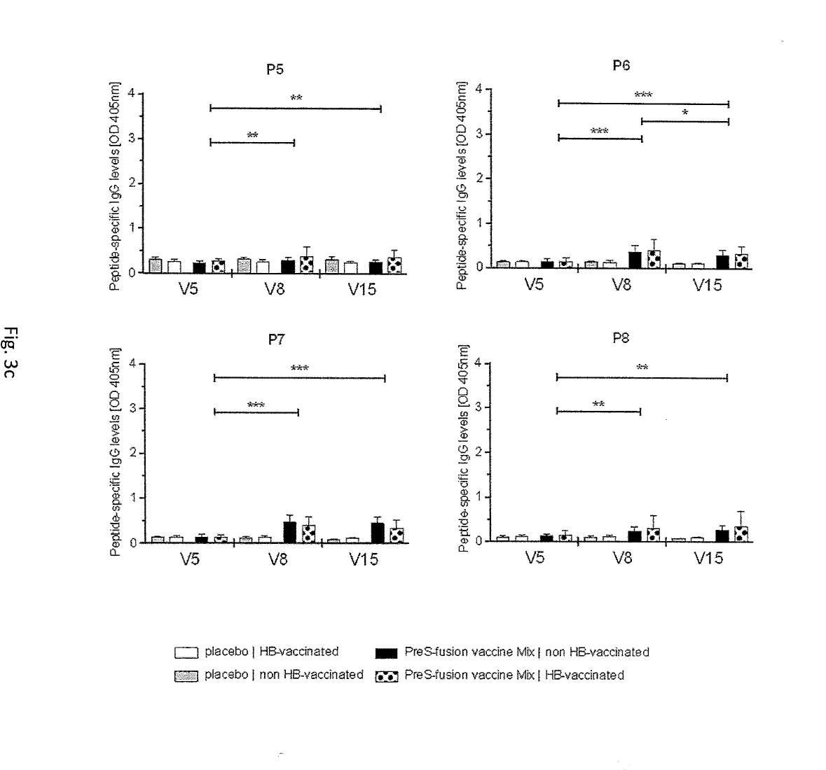

FIG. 4 shows PreS-specific antibody responses of subjects vaccinated with PreS-fusion vaccine Mix (PreS-FVM) or placebo and antibodies present in hepatitis B-infected individuals. Shown are optical density values (y-axes: OD values) corresponding to IgA, IgE, IgM, IgG and IgG subclass (IgG1-IgG4) levels specific for PreS of subjects immunized with placebo (n=8), 20 μg (n=10) or 40 μg of PreS-fusion vaccine Mix (n=12) as well as of hepatitis B-infected individuals (n=19) (x-axes). Graphs show mean values with SD. Significant differences are indicated: ***p<0.001.

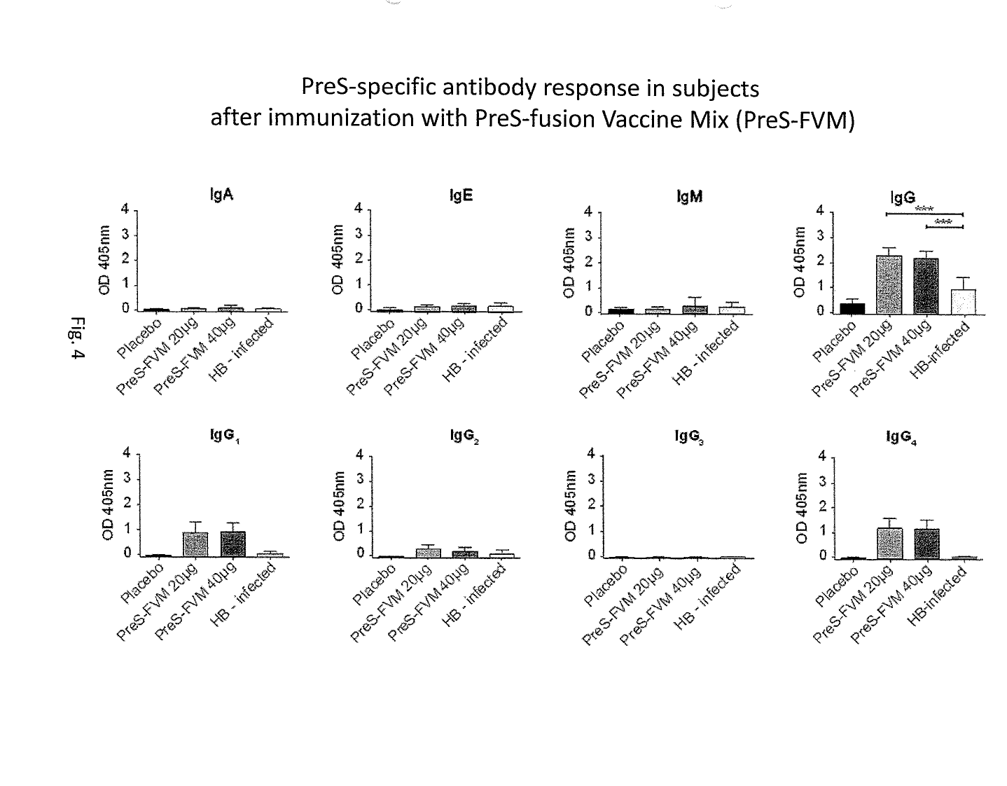

FIG. 5 shows IgG responses specific for PreS peptides P1-P8 of subjects vaccinated with PreS-fusion vaccine Mix (PreS-FVM) or placebo and IgG present in hepatitis B-infected individuals. Shown are optical density values (y-axes: OD values) corresponding to IgG levels specific for PreS-derived peptides (P1-P8) of subjects immunized with placebo (n=8), 20 μg (n=10) or 40 μg of PreS vaccine mixture (n=12) at V15 as well as of hepatitis B-infected individuals (n=19) (x-axes). Results are represented as mean values with SD.

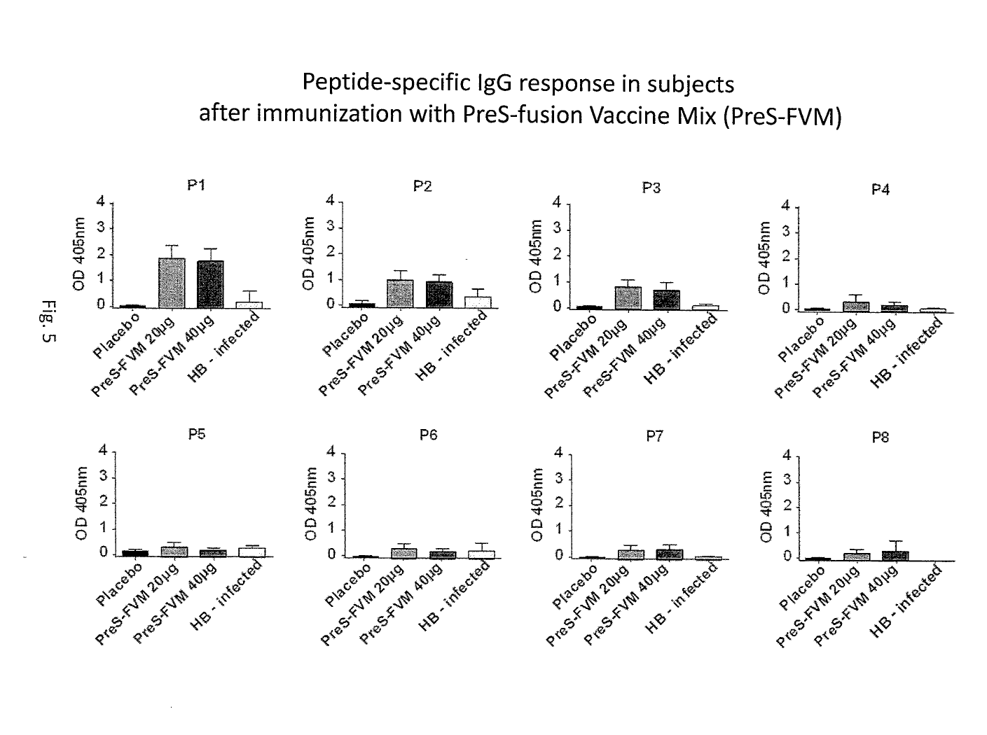

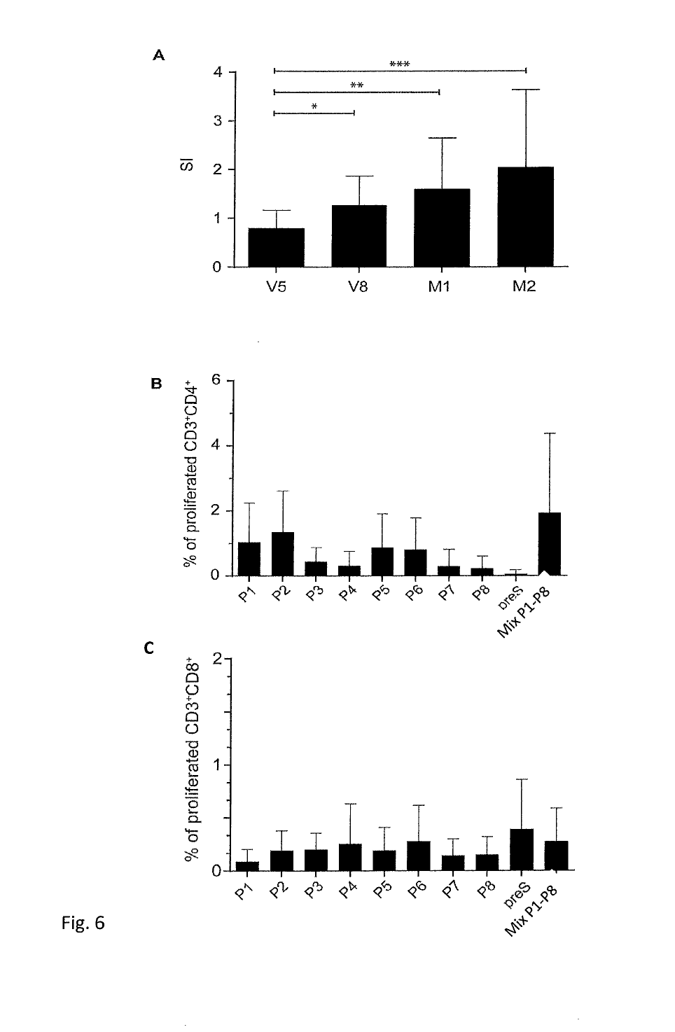

FIG. 6 shows PreS- and peptide-specific T cell responses. FIG. 6A: PreS-specific PBMC proliferations (y-axis: stimulation indices SIs) assessed by [3H] thymidine incorporation in subjects immunized with PreS-fusion vaccine Mix (n=19) at different time points (x-axis). Mean values with SD and significant differences are indicated: *p<0.05, **p<0.01, ***p<0.001. FIG. 6B, and FIG. 6C: Percentages of proliferated CD4 (B) and CD8 (C) T cells (y-axes) after stimulation with PreS peptides (P1-P8), PreS or an equimolar peptide mix (x-axes) in blood samples of subjects immunized with PreS-fusion vaccine Mix (n=11) at time point M2. Results are represented as mean values with SD.

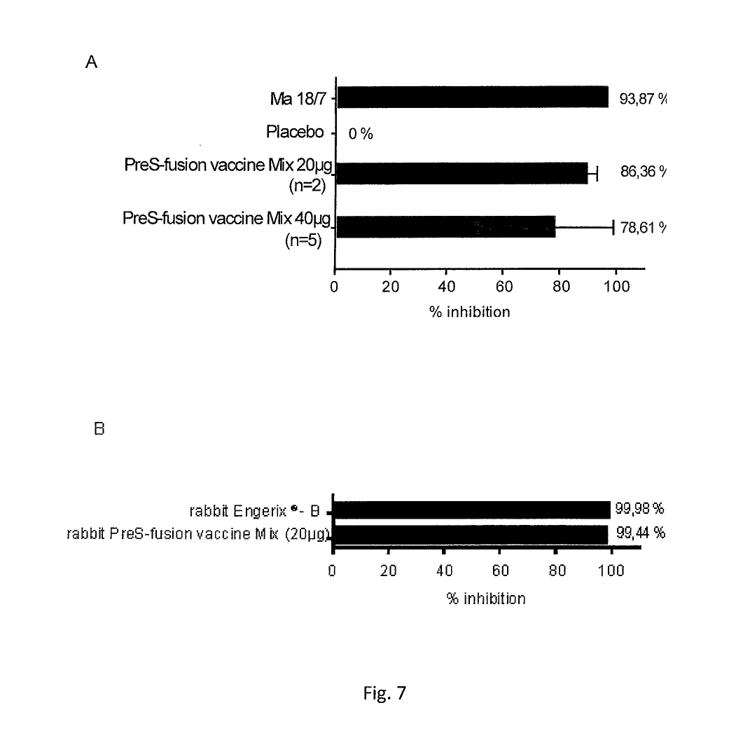

FIG. 7 shows the antibody-induced inhibition of hepatitis B virus infection in an in-vitro virus neutralization assay which is based on in-vitro cultured liver cells. Percentages of the inhibition of hepatitis B infection of cultured HepG2-hNTCP (x-axis) achieved by pre-incubation of virus with anti-sera containing virus neutralizing antibodies. FIG. 7A: Inhibition of virus infection by Ma 18/7 (Positive control), serum from a placebo-treated human subject, sera from human subjects after immunization with PreS-fusion vaccine Mix (n=7), all subjects without prior hepatitis B vaccination. FIG. 7B: Inhibition of virus infection by sera from rabbits immunized with the commercial hepatitis B vaccine Engerix or the PreS-fusion vaccine Mix.

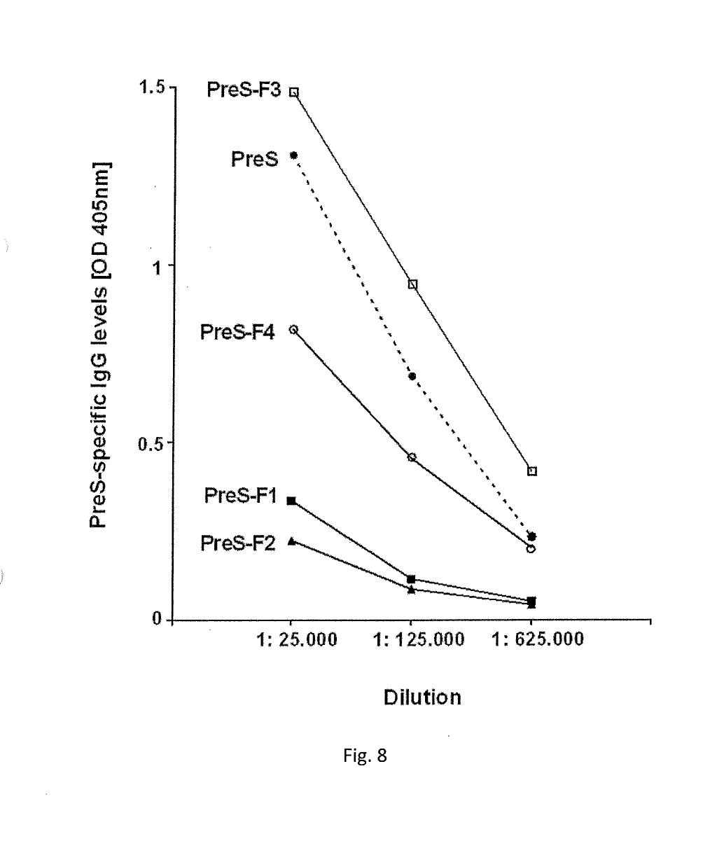

FIG. 8 shows a comparison of total serum IgG towards PreS in sera of New Zealand White (NZW) rabbits which have undergone immunization, either with recombinant PreS or PreS-fusion-proteins (PreS-F1-PreS-F4), as emulsion in Complete Freund's Adjuvant. The x-axis indicates the dilution of sera and on the y-axis, the OD values, measured at 405 nm are depicted. The experiment was assayed in duplicates.

### EXAMPLES

### Example 1: Expression and Purification of Recombinant PreS, Synthesis of PreS Overlapping Peptides, Sequence Alignments

Expression and purification of a hexahistidine-tagged recombinant PreS protein (PreS1+PreS2 (SEQ ID No. 5; genotype A; subtype adw2, derived from GenBank: AAT28735.1) in Escherichia coli BL21 (DE3, Stratagene, USA) has been performed as described in Niespodziana K et al. (J Allergy Clin Immunol 127(2011):1562-70).

Eight peptides of a length of approximately 30 amino acids and an overlap of 10 amino acids spanning the complete sequence of PreS (genotype A, subtype adw2; Table A; FIG. 1) were synthesized by a Fmoc (9-fluorenylmethoxycarbonyl)-strategy with HBTU [2-(1H-Benzotriazol-1-yl)1,1,3,3 tetramethyluronium hexafluorophosphat] activation (CEM-Liberty, Matthews, N.C.; Applied Biosystems, Life technologies, USA).

TABLE A

| Peptide | Sequence | SEQ ID No. |
|---|---|---|
| P1 | GGWSSKPRKGMGTNLSVPNPLGFFPDHQLD | 14 |
| P2 | 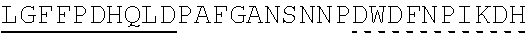 | 15 |
| P3 | 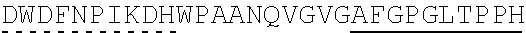 | 16 |
| P4 | 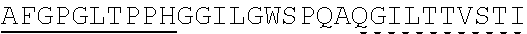 | 17 |
| P5 | 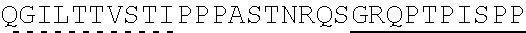 | 18 |
| P6 | 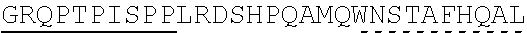 | 19 |
| P7 | 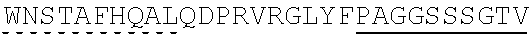 | 20 |
| P8 | PAGGSSSGTVNPAPNIASHISSISARTGDPVTN | 21 |

(overlapping regions of the peptides are underlined)

Peptides were purified by preparative HPLC and their identity was confirmed by mass spectrometry (Microflex MALDI-TOF, Bruker, USA).

An alignment of the PreS genotype A, serotype adw2 sequence and peptide sequences thereof with HBV genotypes B-H was performed with CLUSTAL W using reference sequences from the HBV data base (HBVdb: https://hbvdb.ibcp.fr/HBVdb/HBVdbIndex) (Hayer J et al. Nucleic Acids Res 2012;gks1022) (see FIG. 1).

### Example 2: Immunization of Rabbits

Specific rabbit antibodies against recombinant PreS were raised by immunization of a New Zealand white rabbit with purified PreS (200 μg per injection) using Freund's complete adjuvant (CFA) for the first and incomplete Freund's adjuvant (IFA) for the second and third injection (Charles River, Germany). In addition, New Zealand white rabbits were immunized three times with a mix containing 20 μg (n=2) or 40 μg (n=2) of each of the four PreS vaccine mixture components (PreS vaccine mixture-20/PreS vaccine mixture-40) using Al(OH)₃ as adjuvant. The four PreS vaccine mixture components include PreS fusion proteins PreSF1, PreSF2, PreSF3 and PreSF4 having the following amino acid sequences:

PreSF1 (SEQ ID No. 22):
MVRYTTEGGTKTEAEDVIPEGWKADTSYESKVRYTTEGGTKTEAEDVIPE
GWKADTSYESKGGWSSKPRKGMGTNLSVPNPLGFFPDHQLDPAFGANSNN
PDWDFNPIKDHWPAANQVGVGAFGPGLTPPHGGILGWSPQAQGILTTVST
IPPPASTNRQSGRQPTPISPPLRDSHPQAMQWNSTAFHQALQDPRVRGLY
FPAGGSSSGTVNPAPNIASHISSISARTGDPVTNVRYTTEGGTKTEAEDV
IPEGWKADTSYESKVRYTTEGGTKTEAEDVIPEGWKADTSYESK
PreSF2 (SEQ ID No. 23):
MFRFLTEKGMKNVFDDVVPEKYTIGATYAPEEFRFLTEKGMKNVFDDVVP
EKYTIGATYAPEEGGWSSKPRKGMGTNLSVPNPLGFFPDHQLDPAFGANS
NNPDWDFNPIKDHWPAANQVGVGAFGPGLTPPHGGILGWSPQAQGILTTV
STIPPPASTNRQSGRQPTPISPPLRDSHPQAMQWNSTAFHQALQDPRVRG
LYFPAGGSSSGTVNPAPNIASHISSISARTGDPVTNFRFLTEKGMKNVFD
DVVPEKYTIGATYAPEEFRFLTEKGMKNVFDDVVPEKYTIGATYAPEE
PreSF3 (SEQ ID No. 6):
MEAAFNDAIKASTGGAYESYKFIPALEAAVKAEEVKVIPAGELQVIEKVD
AAFKVAATAANAAPANDKGGWSSKPRKGMGTNLSVPNPLGFFPDHQLDPA
FGANSNNPDWDFNPIKDHWPAANQVGVGAFGPGLTPPHGGILGWSPQAQG
ILTTVSTIPPPASTNRQSGRQPTPISPPLRDSHPQAMQWNSTAFHQALQD
PRVRGLYFPAGGSSSGTVNPAPNIASHISSISARTGDPVTNADLGYGPAT
PAAPAAGYTPATPAAPAEAAPAGKATTEEQKLIEKINAGFKAALAAAAGV
QPADKYR
PreSF4 (SEQ ID No. 24):
MGKATTEEQKLIEDVNASFRAAMATTANVPPADKGKATTEEQKLIEDVNA
SFRAAMATTANVPPADKGGWSSKPRKGMGTNLSVPNPLGFFPDHQLDPAF
GANSNNPDWDFNPIKDHWPAANQVGVGAFGPGLTPPHGGILGWSPQAQGI
LTTVSTIPPPASTNRQSGRQPTPISPPLRDSHPQAMQWNSTAFHQALQDP
RVRGLYFPAGGSSSGTVNPAPNIASHISSISARTGDPVTNGKATTEEQKL
IEDVNASFRAAMATTANVPPADKGKATTEEQKLIEDVNASFRAAMATTAN
VPPADK

Furthermore, rabbit antibodies specific for the registered hepatitis B vaccine ENGERIX-B were obtained by immunizing New Zealand white rabbits (n=2) three-times with commercially available ready-to-use pre-filled syringes at an interval of one month.

Serum samples were obtained before immunization and approximately four weeks after the third immunization and stored at −20° C. until analysis.

Immunization with PreS vaccine mixture showed induction of IgG antibodies with specificity for sequential PreS epitopes. FIG. 2 shows a comparison of the IgG antibody responses towards PreS and synthetic PreS-derived peptides induced in rabbits with CFA-formulated PreS or aluminium hydroxide-adsorbed PreS vaccine mixture (FIG. 2B). Rabbit antibodies induced with CFA-formulated PreS recognized PreS and each of the PreS-derived peptides except of P7 (FIG. 2A). Aluminium-hydroxide adsorbed PreS vaccine mixture in a 20 μg dose induced PreS-specific IgG antibodies and IgG antibodies directed mainly to the N-terminal peptides P1, P2, peptide P6 and towards the C-terminal peptide P8 (FIG. 2B). No PreS or peptide-specific IgG responses were found in rabbits before immunization (FIG. 2, left bars).

### Example 3: Assessment of PreS- and PreS Peptide-Specific Humoral Immune Responses

Serum samples were obtained from patients who have received three injections of Al(OH)₃-adsorbed PreS vaccine mixture (i.e., mixes of 10, 20 or 40 μg of each PreS vaccine mixture component or placebo, i.e., Al(OH)₃). Sera were collected before and four weeks after the third immunization and stored at −20° C. until use. A second set of serum samples was obtained from patients who were treated over a period of two years with seven subcutaneous injections of Al(OH)₃-adsorbed PreS vaccine mixture (i.e., mixes of 20 or 40 μg of each PreS vaccine mixture component or Al(OH)₃ as placebo). In addition, serum samples were obtained from patients suffering from hepatitis B infection which was diagnosed based on clinical data, liver function testing and HBV serum markers.

All sera analyzed, were screened for serological markers for HBV (i.e., hepatitis B surface antigen [HBsAg]; antibodies to the hepatitis B surface antigen [anti-HBs] as well as antibodies to the hepatitis B core antigen [anti-HBc].

ELISA plates (NUNC MaxiSorp®, Denmark) were coated with the antigens (recombinant PreS, synthetic PreS-overlapping peptides: P1-P8) or human serum albumin (negative control) (Behring, USA). Incubation was performed with rabbit sera in a dilution of 1:10,000 (CFA) or 1:500 (PreS vaccine mixture-20/PreS vaccine mixture-40), with mouse sera in a dilution of 1:1,000 and with human sera diluted differently for the isotypes and IgG subclasses. For the detection of human total IgG, sera were diluted 1:100, for IgA, IgG1, IgG2, IgG3, IgG4 as well as IgM, sera were diluted 1:20 and for detection of IgE antibodies sera were diluted 1:10.

Rabbit IgG was detected with donkey anti-rabbit horse radish peroxidase-conjugated IgG antibodies, diluted 1:2,500 (GE Healthcare, Buckinghamshire, Great Britain). Bound mouse IgG1 was detected with monoclonal rat anti-mouse IgG1 (BD Pharmingen, USA) diluted 1:1,000, followed by horse radish peroxidase-conjugated goat anti-rat IgG antibodies (Amersham Bioscience, Sweden) diluted 1:2,500.

Human IgG was detected with rabbit anti-human IgG Fc-specific antibody (Jackson-Dianova, Germany) diluted 1:10,000, followed by peroxidase-linked donkey anti-rabbit IgG (GE Healthcare) at a dilution of 1:2,500. Human IgA, IgG subclasses IgG1, IgG2 and IgG4 as well as human IgM were detected with purified mouse anti-human IgA1/IgA2, IgG1, IgG2, IgG4 and IgM (BD Pharmingen) antibodies, diluted 1:1,000 respectively, followed by peroxidase-linked sheep anti mouse IgG (GE Healthcare) at a dilution of 1:2,500. Monoclonal anti-human IgG3 (Sigma Aldrich, USA) was diluted 1:5,000. Human IgE was detected with goat anti-human horse radish peroxidase-conjugated IgE antibodies (KPL, USA).

### Example 4: PreS-Specific Antibody Responses of PreS Vaccine Mixture Immunized Subjects are not Influenced by Prior Hepatitis B Immunity

Serum samples from human subjects who received immunotherapy with PreS vaccine mixture or with placebo were tested for IgG reactivity to PreS and synthetic PreS peptides (FIGS. 3a to 3c). These patients (n=30) had been screened for hepatitis B-specific serum markers (HBsAg, anti-HBs and anti-HBc antibodies) before treatment and found to be negative for HBsAg and anti-HBc antibodies. Due to previous vaccination with a hepatitis B vaccine, twenty-two of the subjects contained anti-HBs antibodies (FIGS. 3a to 3c). It was found that each of the patients who received immunotherapy with PreS vaccine mixture, regardless if they had been HB-vaccinated before or not, but not placebo-treated patients developed robust PreS-specific IgG responses when sera were tested after the third (V8; three months after first injection) as well as after the seventh injection (V15; 15 months after first injection) (FIGS. 3a to 3c). The PreS-specific IgG responses increased significantly from baseline before immunotherapy (i.e., V5 versus V8) and further increased significantly between V8 and V15 (i.e., after the seventh injection) (FIGS. 3a to 3c). The PreS-specific IgG responses in these patients were directed mainly towards the N-terminal peptides P1, P2 and P3 and again P1- and P2-specific IgG responses showed significant increases from baseline V5 to V8 and from V8 to V15 (FIGS. 3a to 3c). Also increases of IgG responses against the other PreS-derived peptides P4, P5, P6, P7 and P8 were found in sera from patients who received immunotherapy with PreS vaccine mixture but not in placebo-treated patients (FIGS. 3a to 3c).

### Example 5: PreS-Specific Antibody Responses of PreS Vaccine Mixture Immunized Subjects are Directed Against Neutralizing Epitopes and Differ from Those of Hepatitis B-Infected Individuals

FIG. 4 shows a comparison of the PreS-specific isotype and IgG subclass responses of patients after immunotherapy with PreS vaccine mixture or placebo with that of hepatitis B-infected individuals. Immunotherapy with both doses of PreS vaccine mixture induced a robust Pres-specific IgG response in each of the treated patients which was significantly higher than the IgG response in hepatitis B-infected individuals (FIG. 4). No relevant PreS-specific IgA, IgE or IgM responses were detected in sera from patients who were treated with PreS vaccine mixture or placebo as well as in hepatitis B-infected individuals (FIG. 4). The PreS-specific IgG subclass response was different between PreS vaccine mixture-treated subjects and hepatitis B-infected individuals. PreS vaccine mixture-treated subjects showed a preferential IgG1 and IgG4 subclass response to PreS whereas hepatitis B-infected individuals mounted some IgG1 and IgG2 responses towards PreS (FIG. 4).

Also striking differences regarding the epitope specificity of PreS-specific antibodies in PreS vaccine mixture-treated patients versus hepatitis B-infected individuals were found (FIG. 5). PreS vaccine mixture-immunized patients but not hepatitis B-infected individuals showed strong IgG responses towards P1 and P3 (FIG. 5). This finding is surprising because the region defined by P1 corresponds to a motif within PreS1 (see also FIG. 1) that has been reported to contain the essential residues for inhibition of hepatitis B-infections. Furthermore, P7 was recognized only by PreS vaccine mixture-treated subjects but not by hepatitis B-infected individuals whereas IgG responses towards P2 and P6 were also found in hepatitis B-infected individuals (FIG. 5).

### Example 6: Assessment of T Cell Responses

Peripheral Blood Mononuclear Cells (PBMC) were obtained from heparinized blood samples through density gradient centrifugation using Ficoll (Amersham Biosciences, Sweden). When blood samples could be obtained, PreS-specific PBMC proliferation was determined in PreS vaccine mixture-vaccinated subjects (n=19) at V5, V8, M1 (5 months after first vaccination) and M2 (17 months after first vaccination) by [3H]-thymidine incorporation.

For certain PreS vaccine mixture-immunized patients (n=11) CD4 and CD8 T cell responses could be assessed at M2 by carboxyfluorescein succinimidyl ester (CFSE) labelling.

Fluorescent dye-labelled cells were seeded at 200,000 cells/well in Ultra Culture™ serum-free medium (Lonza, Belgium) supplemented with 2 mmol/L L-glutamine (Sigma Aldrich, USA), 50 mmol/L β-mercaptoethanol (Sigma Aldrich), and 0.02 mg of gentamicin per milliliter (Sigma Aldrich), in a total volume of 200 μl in 96 well microplates with U shaped bottom (Thermo Fisher, USA). Cells were either left unstimulated (negative control) or were stimulated with Dynabeads® Human T-Activator CD3/CD28 (3 μg/well (Invitrogen, USA)) as positive control or with PreS (0.15 μg/well), equimolar quantities of PreS-overlapping peptides (0.03 μg/well) or with a mixture of the PreS-derived overlapping peptides containing 0.03 μg/well of each peptide and cultured at 37° C. in 5% CO₂ for 7 days before antibody staining and FACS analysis was conducted.

For flow cytometry the following reagents were used: PerCP/Cy5.5 anti-human CD3 antibody (Clone HIT3a), Brilliant Violet 421™ anti-human CD4 antibody (Clone RPA-T4), APC anti-human CD8a antibody (Clone HIT8a), as well as isotype controls, i.e., PerCP/Cy5.5 mouse IgG2a, Brilliant Violet 421™ mouse IgG1, APC mouse IgG1 (BioLegend, USA) and Fixable Viability Dye EFluor™ 780 (eBioscience, USA).

Flow Cytometry was performed on a BD FACS Canto II (Becton, Dickinson and Company, USA). Twenty thousand events were acquired per sample and analysis was performed via FlowJo Software, Version 10. Lymphocytes were gated according to morphological criteria on a forward and sideward scatter dot blot, dead cells were excluded by staining of viability dye and gating was focused on CD3CD4 and CD3CD8-positive T cells. Those cells that proliferated in response to antigen stimulation were identified by their reduction in CFSE fluorescence intensity. Results represent means of triplicate cultures and 235 median percentages stimulation of CD3+CD4+ and CD3+CD8+ above background are shown for the different antigens and the analysed patients.

FIG. 6 shows the development of PreS-specific T cell responses in patients who received immunotherapy with PreS vaccine mixture. A gradually increasing PreS-specific T cell response was found which was significantly higher at V8, M1 and M2 as compared to baseline at V5 (FIG. 6A). When the epitope specificity of the PreS-specific CD4 cell responses was analyzed by CFSE staining we found that P1, P2, P5 and P6 induced the strongest CD4 cell proliferation but CD4 responses towards P3, P4 and P7 were also found (FIG. 6B). Interestingly, the peptides and the peptide mix induced stronger CD4 cell proliferation than the PreS protein (FIG. 6B). Albeit at low frequency, some PreS and PreS peptide-specific CD8 cell response was detected which was mainly directed towards P2, P3, P6 and P8 and complete PreS (FIG. 6B).

### Example 7: Hepatitis B Virus Neutralization Assays

The HBV inoculum for infection was prepared from supernatants of HepAd38 cells using a heparin column (GE Healthcare, Great Britain) to isolate viral particles. HepG2-hNTCP cells20 were seeded at a density of 3×10⁵ cells/well in a 24 well plate. At day two after seeding, the infection medium (DMEM, Invitrogen, USA) was supplemented with 2.5% DMSO (Merck, Germany) and at day three cells were infected with HBV. For the neutralization of HBV particles, patients' sera (10 μl) were pre-incubated with the HBV inoculum (6.9×10⁷ genome equivalents (GE)/well) for 30 minutes at 37° C., followed by co-incubation of cells with the patients' sera and virus in presence of 4% polyethylene glycol 800 (Sigma Aldrich, USA) for 16 hours at 37° C. The neutralizing monoclonal antibody Ma18/721 was used as positive control.

After 16 hours of inoculation, cells were washed extensively with PBS and fresh differentiation medium, supplemented with 2.5% DMSO (Invitrogen) was added. Additional medium changes were performed at day three and day five post infection.

Quantification of HBV infection was conducted by the measurement of secreted hepatitis B e antigen (HBeAg) in the supernatant from cells at day five to seven after infection. HBeAg was determined by ADVIA Centaur XPT automated chemiluminescence system (Siemens, Germany). Samples were considered as positive at a signal above 1 Index.

The expression of HBV core protein was detected by specific immunofluorescence. The supernatant was removed and the cells were washed with PBS prior to the fixation with 4% paraformaldehyde (Sigma Aldrich) for 30 minutes at room temperature (RT). Next, cells were washed with PBS followed by the permeabilization with 0.25% Triton X 100 (AppliChem GmbH, Germany) in PBS for 30 minutes at RT. Then, cells were incubated overnight at 4° C. with the primary antibody (anti-HBV core, rabbit polyclonal AK, DAKO Deutschland GmbH, Hamburg, Germany) diluted in 2% w/v BSA, PBS. On the next day, cells were washed with PBS and finally incubated with the secondary antibody (goat a rabbit Alexa 488; Invitrogen, Carlsbad, Calif.) and 4′, 6-Diamidin-2-phenylindo/Hoechst 33342 (Roche Applied Science, Germany) in the dark. For the detection of HBV core protein, the secondary antibody was incubated for 2 hours at RT, protected from light. Cells were examined under fluorescence microscope using 480 nm for Alexa-488-labeled secondary antibodies (Invitrogen, Carlsbad, Calif.) and 360 nm for the nuclear staining.

In the first type of assay the expression of hepatitis B core antigen (HBcAg) after infection of cells is detected by specific immunofluorescence. No HBcAg has been detected in uninfected cells but in infected and untreated cells and that expression can be prevented by pre-incubation of virus with the neutralizing monoclonal antibody Ma18/721 which is directed against the PreS1 domain of the large hepatitis B surface protein. Likewise it was found that pre-incubation of hepatitis B virus with rabbit antibodies induced by the commercial vaccine Engerix-B or with rabbit anti-PreS vaccine mixture (20 μg dose) antibodies inhibited infection of HepG2-hNTCP cells. A similar set of experiments was performed with sera from PreS vaccine mixture- or placebo-treated patients. Sera obtained from a patient before and after immunization with placebo did not inhibit infection of HepG2-hNTCP cells whereas sera obtained from a patient after immunization with 20 μg or from a patient after immunization with 40 μg inhibited infection of HepG2-hNTCP cells.

In addition to the staining of the HBcAg an assay based on the measurement of secreted hepatitis B e antigen (HBeAg) by HepG2-hNTCP cells was used seven days post infection with HBV as another surrogate marker to quantify the inhibition of HBV infection. It was found that sera from PreS vaccine mixture-treated inhibited HBV infection between 50-99% (FIG. 7A). No relevant difference was found depending on the dose and number of PreS vaccine mixture injections because a similar inhibition was observed for sera from patients who had received three injections (FIG. 7A) as well as for sera from patients who had received seven injections (FIG. 7A, black). Also, there was no obvious difference regarding the degree of inhibition between patients who either received the 20 μg or 40 μg dose of PreS vaccine mixture (FIG. 7A). No inhibition was observed for serum from a placebo treated patient and a more than 90% inhibition was observed for the monoclonal antibody Ma 18/7 (FIG. 7A). Rabbit anti-Engerix-B and rabbit anti-PreS vaccine mixture antibodies caused a more than 99% inhibition of HBV infection (FIG. 7B).

### Example 8

Recombinant PreS and human serum albumin (Behring, USA) as negative control were coated onto Nunc Maxisorb microplates (Thermo-Fisher Scientific, USA) at a concentration of 2 μg/ml in 100 mM sodium phosphate buffer, pH 9.6 overnight at 4° C. Wash buffer was comprised of PBS, 0.05% v/v Tween20 (PBS/T) and the blocking procedures were performed with 2% w/v BSA, PBS/T for 2 hours at 37° C. All subsequent serum and reagent dilutions were done in 0.5% w/v BSA, PBS/T.

To determine humoral immune responses of rabbits, which have undergone complete immunization, either with recombinant PreS or PreS-fusion-proteins, as emulsion in Complete Freund's Adjuvant (CFA), sera were used in different dilutions (4° C., overnight) and bound total rabbit IgG was detected using donkey anti-rabbit horse radish peroxidase-conjugated IgG antibodies diluted 1:2.000 (GE Healthcare, Great Britain). The color reaction was induced by ABTS [2, 2′-azino-bis (3-ethylbenzothiazoline-6-sulphonic acid] and absorbance detection, corresponding to the levels of antigen-specific antibodies was performed at 405 nm and 490 nm using a microplate reader (Molecular Devices, USA). All determinations were performed in triplicates.

It surprisingly turned out that only a fusion protein comprising one or more peptides having the amino acid sequences SEQ ID No. 1, SEQ ID No. 2, SEQ ID No. 3 and/or SEQ ID No. 4 and PreS (PreS-F3) were able to induce the formation of PreS specific IgG to a much higher extend compared to PreS alone or other fusion proteins comprising also PreS fused to different peptides (PreS-F1, PreS-F2, PreS-F4) as depicted in FIG. 8.

1. Fusion protein for use in the treatment and/or prevention of a hepatitis B virus infection comprising at least one hepatitis B PreS polypeptide or fragment thereof fused to at least one peptide consisting of an amino acid sequence having at least 80% identity to a sequence selected from the group consisting of SEQ ID No. 1, SEQ ID No. 2, SEQ ID No. 3 and SEQ ID No. 4.

2. Fusion protein for the use according to claim 1, wherein the amino acid sequence of the PreS polypeptide is at least 80% identical to SEQ ID No. 5, SEQ ID No. 7, SEQ ID No. 8, SEQ ID No. 9, SEQ ID No. 10, SEQ ID No. 11, SEQ ID No. 12 or SEQ ID No. 13.

3. Fusion protein for the use according to claim 1, wherein the at least one peptide is fused to the N- and/or C-terminus of the PreS polypeptide.

4. Fusion protein for the use according to claim 1, wherein the fusion protein comprises an amino acid sequence which is at least 80% identical to SEQ ID No. 6.

5. Fusion protein for the use according to claim 1, wherein the hepatitis B virus infection is caused by a hepatitis B virus genotype A, B, C, D, E, F, G, H or a subtype thereof.

6. Fusion protein for the use according to claim 1, wherein the fusion protein is administered to an individual at least once in an amount of 0.01 μg/kg body weight to 5 mg/kg body weight, preferably 0.1 μg/kg body weight to 2 mg/kg body weight.

7. Fusion protein for the use according to claim 1, wherein the fusion protein is administered together with at least one adjuvant and/or pharmaceutical acceptable excipient.


SEQUENCE LISTING (24 sequences)

SEQ ID NO: 1 - protein, 30 aa, Artificial Sequence
  Artificial peptide
EAAFNDAIKASTGGAYESYKFIPALEAAVK

SEQ ID NO: 2 - protein, 37 aa, Artificial Sequence
  Artificial peptide
AEEVKVIPAGELQVIEKVDAAFKVAATAANAAPANDK

SEQ ID NO: 3 - protein, 33 aa, Artificial Sequence
  Artificial peptide
ADLGYGPATPAAPAAGYTPATPAAPAEAAPAGK

SEQ ID NO: 4 - protein, 33 aa, Artificial Sequence
  Artificial peptide
ATTEEQKLIEKINAGFKAALAAAAGVQPADKYR

SEQ ID NO: 5 - protein, 173 aa, Hepatitis B virus
GGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTN

SEQ ID NO: 6 - protein, 307 aa, Artificial Sequence
  PreSF3
MEAAFNDAIKASTGGAYESYKFIPALEAAVKAEEVKVIPAGELQVIEKVDAAFKVAATAANAAPANDKGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNADLGYGPATPAAPAAGYTPATPAAPAEAAPAGKATTEEQKLIEKINAGFKAALAAAAGVQPADKYR

SEQ ID NO: 7 - protein, 173 aa, Hepatitis B virus
GGWSSKPRKGMGTNLSVPNPLGFFPDHQLDPAFKANSDNPDWDLNPHKDNWPDSNKVGVGAFGLGFTPPHGGLLGWSPQAQGILTTVPAAPPPASTNRQVARPPTPLSPPLRDTHPQAMQWNSTTFHQTLQDPRVRALYFPAGGSSSGTVNPVQNTASSISSILSKTGDPVPN

SEQ ID NO: 8 - protein, 173 aa, Hepatitis B virus
GGYSSKPRKGMGTNLSVPNPLGFLPDHQLDPAFGANSNNPDWDFNPNKDPWPEAWQVGVGAFGPGFTPPHGGLLGWSPQAQGILTPVPAAPPPASTNRQSGRQPTPISPPLRDSHPQAMQWNSTTFHQALLDPRVRGLYFPAGGSSSGTTNPVPTTASPISSIFSRTGDPAPN

SEQ ID NO: 9 - protein, 162 aa, Hepatitis B virus
GQNLSTSNPLGFFPDHQLDPAFRANTANPDWDFNPNKDTWPDANKVGAGAFGLGFTPPHGGLLGWSPQAQGILQTLPTNPPPASTNRQSGRQPTPLSPPLRNTHPQAMQWNSTTFHQTLQDPRVRGLYFPAGGSSSGTVNPVPTTVSHISSIFSRIGDPALN

SEQ ID NO: 10 - protein, 172 aa, Hepatitis B virus
GLSWTVPLEWGKNHSTTNPLGFFPDHQLDPAFRANTRNPDWDHNPNKDHWTAANKVGVGAFGPGFTPPHGGLLGWSPQAQGMLKTLPADPPPASTNRQSGRQPTPITPPLRDTHPQAMQWNSTTFHQALQDPRVRGLYFPAGGSSSGTVNPVPTTASLISSIFSRIGDPAPN

SEQ ID NO: 11 - protein, 173 aa, Hepatitis B virus
GAPLSTTRRGMGQNLSVPNPLGFLPDHQLDPLFRANSSSPDWDFNKSKDNWPMANKVGVGGYGPGFTPPHGGLLGWSPQAQGVLTTLPADPPPASTNRRSGRKPTPVSPPLRDTHPQAMQWNSTQFHQALLDPRVRALYFPAGGSSSETQNPAPTIASLTSSIFLKTGGPAMN

SEQ ID NO: 12 - protein, 172 aa, Hepatitis B virus
GLSWTVPLEWGKNLSTSNPLGFLPDHQLDPAFRANTNNPDWDFNPKKDPWPEANKVGVGAYGPGFTPPHGGLLGWSPQSQGTLTTLPADPPPASTNRQSGRQPTPISPPLRDSHPQAMQWNSTAFHQALQNPKVRGLYFPAGGSSSGIVNPVPTIASHISSIFSRIGDPAPN

SEQ ID NO: 13 - protein, 173 aa, Hepatitis B virus
GAPLSTARRGMGQNLSVPNPLGFFPDHQLDPLFRANSSSPDWDFNTNKDNWPMANKVGVGGFGPGFTPPHGGLLGWSPQAQGILTTSPPDPPPASTNRRSGRKPTPVSPPLRDTHPQAMQWNSTQFHQALLDPRVRGLYFPAGGSSSETQNPAPTIASLTSSIFSKTGDPAMN

SEQ ID NO: 14 - protein, 30 aa, Artificial Sequence
  preS fragment
GGWSSKPRKGMGTNLSVPNPLGFFPDHQLD

SEQ ID NO: 15 - protein, 30 aa, Artificial Sequence
  preS fragment
LGFFPDHQLDPAFGANSNNPDWDFNPIKDH

SEQ ID NO: 16 - protein, 30 aa, Artificial Sequence
  preS fragment
DWDFNPIKDHWPAANQVGVGAFGPGLTPPH

SEQ ID NO: 17 - protein, 30 aa, Artificial Sequence
  preS fragment
AFGPGLTPPHGGILGWSPQAQGILTTVSTI

SEQ ID NO: 18 - protein, 30 aa, Artificial Sequence
  preS fragment
QGILTTVSTIPPPASTNRQSGRQPTPISPP

SEQ ID NO: 19 - protein, 30 aa, Artificial Sequence
  preS fragment
GRQPTPISPPLRDSHPQAMQWNSTAFHQAL

SEQ ID NO: 20 - protein, 30 aa, Artificial Sequence
  preS fragment
WNSTAFHQALQDPRVRGLYFPAGGSSSGTV

SEQ ID NO: 21 - protein, 33 aa, Artificial Sequence
  preS fragment
PAGGSSSGTVNPAPNIASHISSISARTGDPVTN

SEQ ID NO: 22 - protein, 294 aa, Artificial Sequence
  PreSF1
MVRYTTEGGTKTEAEDVIPEGWKADTSYESKVRYTTEGGTKTEAEDVIPEGWKADTSYESKGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNVRYTTEGGTKTEAEDVIPEGWKADTSYESKVRYTTEGGTKTEAEDVIPEGWKADTSYESK

SEQ ID NO: 23 - protein, 298 aa, Artificial Sequence
  PreSF2
MFRFLTEKGMKNVFDDVVPEKYTIGATYAPEEFRFLTEKGMKNVFDDVVPEKYTIGATYAPEEGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNFRFLTEKGMKNVFDDVVPEKYTIGATYAPEEFRFLTEKGMKNVFDDVVPEKYTIGATYAPEE

SEQ ID NO: 24 - protein, 306 aa, Artificial Sequence
  PreSF4
MGKATTEEQKLIEDVNASFRAAMATTANVPPADKGKATTEEQKLIEDVNASFRAAMATTANVPPADKGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNGKATTEEQKLIEDVNASFRAAMATTANVPPADKGKATTEEQKLIEDVNASFRAAMATTANVPPADK